(19) United States
(12) Patent Application Publication
(10) Pub. No.: US 2023/0372558 A1
(43) Pub. Date: Nov. 23, 2023

(54) SYSTEMS AND METHODS FOR AUTOMATED DESIGN

(71) Applicant: FERMI RESEARCH ALLIANCE, LLC, Batavia, IL (US)

(72) Inventor: Thomas Kroc, Batavia, IL (US)

(21) Appl. No.: 18/319,376

(22) Filed: May 17, 2023

Related U.S. Application Data
(60) Provisional application No. 63/343,523, filed on May 18, 2022.

(51) Int. Cl. A61L 2/08; A61L 2/26

(52) U.S. Cl. CPC A61L 2/087; A61L 2/082; A61L 2/26; A61L 2202/24

(57) ABSTRACT

A treatment system for treating or sterilizing products includes a particle accelerator configured to produce a beam of charged particles, a deflector configured to redirect the beam of charged particles, a vacuum chamber that prevents the atmosphere from interfering with the charged particles, and a magnet array for directing the beam of charged particles onto a product, wherein the beam of charged particles is used to sterilize the product.

### CROSS REFERENCE TO RELATED PATENT APPLICATIONS

The present application claims the priority and benefit under 35 U.S.C. § 119(e) of U.S. Provisional Patent Application Ser. No. 63/343,523 filed May 18, 2022, entitled “ELECTRON BEAM STERILIZATION.” U.S. Provisional Patent Application Ser. No. 63/343,523 is herein incorporated by reference in its entirety.

### STATEMENT OF GOVERNMENT RIGHTS

The invention described in this patent application was made with Government support under the Fermi Research Alliance, LLC, Contract Number DE-ACO2-07CH11359 awarded by the U.S. Department of Energy. The Government has certain rights in the invention.

### TECHNICAL FIELD

Embodiments are generally related to the field of irradiation including industrial sterilization and other irradiation processes. Embodiments are also related to sterilization of medical devices and other single use systems used for processes such as vaccine and pharmaceutical production. Embodiments further relate to magnetic devices and accelerators that produce electron beams. Embodiments are further related to retrofitting sterilization schemes. Embodiments are also related to material cross liking applications.

### BACKGROUND

Irradiation is a process by which an object may be exposed to radiation. The exposure can originate from various sources, including natural sources. Most frequently, however, the term “irradiation” relates to ionizing radiation, and to a level of radiation that will serve a specific purpose, such as sterilization or processing of materials and structures.

Irradiation can include processes such as sterilization, medical applications, ion implantation, ion irradiation, and industrial chemical applications. Irradiation can use an electron beam itself, or by way of a Bremsstrahlung converter, X-rays. X-rays may be produced by irradiating a target made of a material containing a large proportion of high atomic number atoms or ions with a suitably high-energy electron beam. The X-ray beam is produced by accelerating electrons across a large electric potential difference or electric gradient creating a beam of high-energy electrons and then guiding the beam to the target. The electrons in the electron beam interact with the electric field of the high atomic number nuclei and emit X-ray photons through the Bremsstrahlung process. The generated X-rays have a continuous spectrum, having an upper energy limit determined by the energy of the incident electrons.

Medical device sterilization approaches which presently serve 85% of the existing market are considered “at-risk” by the industry, including gamma radiation sterilization. Uncertainties about the future of these existing approaches may constrain projected growth in the medical device sterilization industry and affect the near-term availability of safe and sterile products.

In general, it is a difficult and time-consuming process for a medical device company to change sterilization modalities. Device material compatibility is a major constraint. However, an historical absence of established processes, data, and know-how in making the change from gamma radiation sterilization have stunted adoption of new, effective, and sustainable methods. The most widely used sterilization modalities, like gamma irradiation and ethylene oxide, are preferred because they benefit from a multiple-decade body of knowledge that affirms safety, efficacy, availability, and affordability of sterilized products.

However, there are major disadvantages to such modalities, the most obvious of which is the increasing pressures on market supply of the cobalt necessary to realize gamma irradiation, in the quantities necessary for future applications. Although cobalt remains viable today, limited supplies suggest in the near future different mechanisms for generating ionizing radiation will be necessary for such sterilization techniques.

As such, there is a need in the art for new sterilization means which can be provided to efficiently and safely harness irradiation for industrial sterilization, treatment of medical devices and other single use systems used for processes such as vaccine and pharmaceutical production as further detailed herein.

### BRIEF SUMMARY

The following summary is provided to facilitate an understanding of some of the innovative features unique to the disclosed embodiments and is not intended to be a full description. A full appreciation of the various aspects of the embodiments disclosed herein can be gained by taking the entire specification, claims, drawings, and abstract as a whole.

It is, therefore, one aspect of the disclosed embodiments to provide for an irradiating system and method.

It is another aspect of the disclosed embodiments to provide a sterilization system and method.

It is another aspect of the disclosed embodiments to provide systems and methods for medical device sterilization.

It is another aspect of the disclosed embodiments to provide systems and methods for the sterilization of single-use systems for pharmaceutical and vaccine production.

It is another aspect of the disclosed embodiments to provide a sterilization system and method making use of electron beam or X-ray radiation.

It is another aspect of the disclosed embodiments to provide retrofit systems for sterilization.

In certain embodiments, methods, and systems for retrofitting gamma radiation sterilization systems, with E-beam and X-ray radiation sterilization is disclosed. E-beam and X-ray radiation sterilization approaches are allowed by regulatory agencies, have an established ISO standard (11137), and have established safety and security benefits.

Due to the absence of established processes, data, and know-how, adoption of X-ray sterilization has suffered despite its acceptance in the pertinent regulations and standards. Furthermore, large scale adoption of E-beam sterilization is lacking.

Sterilization often represents a very small percentage of a medical device's production cost. However, the sterilization step has an outsized impact on the time, efficacy, and ability to get a product to market. The disclosed embodiments provide methods and systems for replacing legacy sterilization approaches that currently occupy the majority of the sterilization market.

The disclosed embodiments are directed to sterilization of safe and accessible medical devices. Such sterilization breeds resilient and scalable sterilization supply chains as required to meet high industry purposes. Among the principal challenges is the supply and security of cobalt-60 for gamma sterilization. The disclosed embodiments can be used to retrofit cobalt-60 based systems and/or to install functionally similar systems where cobalt-60 systems are no longer practical or possible. This allows the embodiments disclosed herein to address new sterilization capacity demands.

Regulatory constraints are a notable hurdle for the use of ionizing radiation in sterilization schemes. Likewise, sterilization is a complex, non-linear process with many interdependencies and decision points across product design, business, and manufacturing functions. These factors and the strategies and processes for navigating them are poorly understood at multiple levels and across functions for medical device companies. The absence of broad and informed understanding of these solutions' capabilities limits industry-informed market and technology development activities.

The use of E-beam and X-rays are established technologies in certain fields, there the embodiments disclosed herein take advantage of increased power and throughput. In particular, accelerator use for medical sterilization as disclosed herein will aid in providing sufficient capacity to meet sterilization demand.

The aforementioned aspects and other objectives and advantages can now be achieved as described herein. In an embodiment, the beam of charged particles can be redirected by the parallelizing permanent magnet array from a diverging pattern output from the scanning electromagnet to a parallel pattern after being subjected to the parallelizing permanent magnet array. In an embodiment, the beam of charged particles can comprise an electron beam or X-ray beam.

In an embodiment, a treatment system, comprises a particle accelerator configured to produce a beam of charged particles, a deflector configured to redirect the beam of charged particles, a vacuum chamber that prevents the atmosphere from interfering with the charged particles, and a magnet array for directing the beam of charged particles onto a product, wherein the beam of charged particles is used to sterilize the product. In an embodiment, the beam of charged particles comprises an electron beam. In an embodiment, the beam of charged particles comprises an X-ray beam. In an embodiment, the treatment system further comprises a target material, wherein the beam of charged particles from the particle accelerator is directed onto the target material to produce Bremsstrahlung X-rays. In an embodiment, the magnet array comprises a parallelizing magnet array for parallelizing the beam of charged particles. In an embodiment, the treatment system further comprises a scan horn protector assembly. The scan horn protector assembly comprises a housing, a concentrator cone, and a sacrificial burst disc at the end of the concentrator cone. In an embodiment of the treatment system, the charged particles are directed onto at least one treatment area.

In an embodiment, a system comprises a particle accelerator configured to produce a beam of electrons, a deflector configured to split the electron beam into a first beam and a second beam, a first re-parallelizing magnet configured to redirect the first beam into a beam bending assembly, wherein the beam bending assembly directs the first beam in a first direction, and a second re-parallelizing magnet configured to redirect the second beam into the beam bending assembly wherein the beam bending assembly directs the second beam in a second direction. In an embodiment, the beam bending assembly comprises a common pole magnet in spaced relation between two opposite pole magnets. In an embodiment, a magnetic field exists between the common pole magnet and opposite pole magnets. In an embodiment, the first direction of the first beam is different from the second direction of the second beam. In an embodiment, the first direction of the first beam is directed to the path of travel of a product on a conveyor assembly and the second direction of the second beam is directed to a different path of travel of a product on the conveyor assembly. In an embodiment, the product comprises a medical product. In an embodiment the system further comprises a target material, wherein the beam of charged particles from the particle accelerator is directed onto the target material to produce Bremsstrahlung X-rays.

In another embodiment, a treatment method comprises producing a particle beam with a particle accelerator, splitting the particle beam into a first particle beam and a second particle beam with a deflector, redirecting the first beam into a beam bending assembly, with a first re-parallelizing magnet, redirecting the second beam into a beam bending assembly with a second re-parallelizing magnet, directing the first particle beam onto a treatment area with a beam bending assembly, and directing the second particle beam onto a different treatment area with the beam bending assembly. In an embodiment, the particle beam comprises an electron beam. In an embodiment, the treatment method comprises producing an x-ray beam by directing the electron beam onto a target. In an embodiment, the treatment method further comprises conveying a product through the treatment area wherein the particle beam is incident on the product. In an embodiment of the method the product comprises a medical product.

### BRIEF DESCRIPTION OF THE FIGURES

The accompanying figures, in which like reference numerals refer to identical or functionally similar elements throughout the separate views and which are incorporated in and form a part of the specification, further illustrate the present invention and, together with the detailed description of the invention, serve to explain the principles of the present invention.

FIG. 1 depicts a perspective cut-away view of RF structures that can form elements of an electron accelerator that can be adapted for use in accordance with a preferred embodiment;

FIG. 2A depicts a perspective cut-away view of a superconducting RF structure that can also form elements of an electron accelerator adapted for use in accordance with an embodiment. The figure indicates the operating principles of such an elliptical RF cavity;

FIG. 2B depicts an accelerator system, in accordance with the disclosed embodiments;

FIG. 3 illustrates a schematic diagram of a magnetic apparatus, in accordance with an embodiment;

FIG. 4A illustrates a schematic diagram of a single accelerator sterilization system, in accordance with an embodiment;

FIG. 4B illustrates a schematic diagram of a multi-accelerator sterilization system, in accordance with an embodiment;

FIG. 5 illustrates a schematic diagram of a vertical accelerator sterilization system, in accordance with an embodiment;

FIG. 6A illustrates aspects of a deflector assembly, in accordance with the disclosed embodiments;

FIG. 6B illustrates aspects of a deflector assembly, in accordance with the disclosed embodiments;

FIG. 6C illustrates aspects of a deflector assembly, in accordance with the disclosed embodiments;

FIG. 7A illustrates a schematic diagram of a scan horn protector assembly, in accordance with the disclosed embodiments;

FIG. 7B illustrates the functional mechanisms associated with the scan horn protector, in accordance with the disclosed embodiments;

FIG. 8A illustrates steps in a method for sterilization, in accordance with the disclosed embodiments;

FIG. 8B illustrates steps in a method for sterilization, in accordance with the disclosed embodiments;

FIG. 9 illustrates a block diagram of a control system, in accordance with the disclosed embodiments;

FIG. 10 depicts a block diagram of a computer system which is implemented in accordance with the disclosed embodiments;

FIG. 11 depicts a graphical representation of a network of data-processing devices in which aspects of the present embodiments may be implemented; and

FIG. 12 depicts a computer software system for directing the operation of the data-processing system depicted in FIG. 11 in accordance with an embodiment.

### DETAILED DESCRIPTION

The particular values and configurations discussed in these non-limiting examples can be varied and are cited merely to illustrate one or more embodiments and are not intended to limit the scope thereof.

Example embodiments will now be described more fully hereinafter with reference to the accompanying drawings, in which illustrative embodiments are shown. The embodiments disclosed herein can be embodied in many different forms and should not be construed as limited to the embodiments set forth herein; rather, these embodiments are provided so that this disclosure will be thorough and complete, and will fully convey the scope of the embodiments to those skilled in the art. Like numbers refer to like elements throughout.

The terminology used herein is for the purpose of describing particular embodiments only and is not intended to be limiting. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of example embodiments in whole or in part.

In general, terminology may be understood at least in part from usage in context. For example, terms, such as “and,” “or,” or “and/or,” as used herein may include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, may be used to describe any feature, structure, or characteristic in a singular sense or may be used to describe combinations of features, structures, or characteristics in a plural sense. In addition, the term “based on” may be understood as not necessarily intended to convey an exclusive set of factors and may, instead, allow for existence of additional factors not necessarily expressly described, again, depending at least in part on context.

Unless otherwise defined, all terms (including technical and scientific terms) used herein have the same meaning as commonly understood by one of ordinary skill in the art. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

U.S. Pat. No. 10,070,509, titled “COMPACT SRF BASED ACCELERATOR,” issued on Sep. 4, 2018, describes a particle accelerator comprising an accelerator cavity, an electron gun, and a cavity cooler configured to at least partially encircle the accelerator cavity. A cooling connector and an intermediate conduction layer are formed between the cavity cooler and the accelerator cavity to facilitate thermal conductivity between the cavity cooler and the accelerator cavity. The embodiments disclosed therein teach a viable, compact, robust, high-power, high-energy electron-beam, or x-ray source. The disclosed advances are integrated into a single design, that enables compact, mobile, high-power electron accelerators. U.S. Pat. No. 10,070,509 is herein incorporated by reference in its entirety.

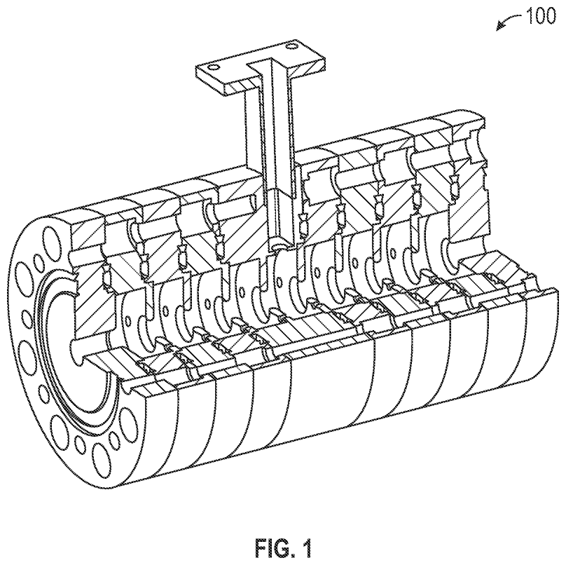

FIG. 1 illustrates a perspective cut-away view of an RF structure 100 that can form elements of an electron accelerator that can be adapted for use in accordance with embodiments disclosed herein. Note that RF accelerator and electron gun structures can be employed to produce electron beams of the required energy for implementation of the disclosed embodiments. An electron accelerator, for example, that employs the RF structure 100 can accelerate electrons generated from an electron gun with RF electric fields in resonant cavities sequenced such that the electrons are accelerated due to an electric field present in each cavity as the electron traverses the cavity.

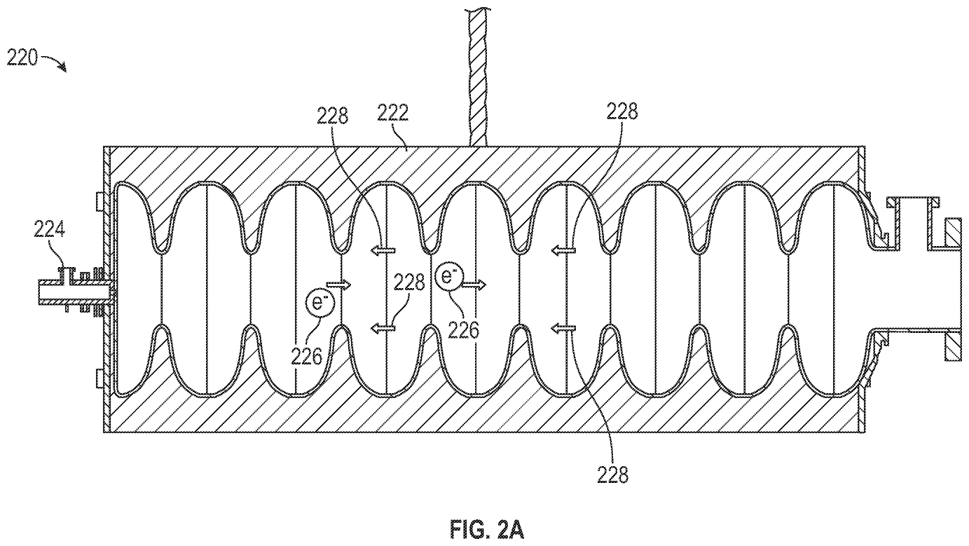

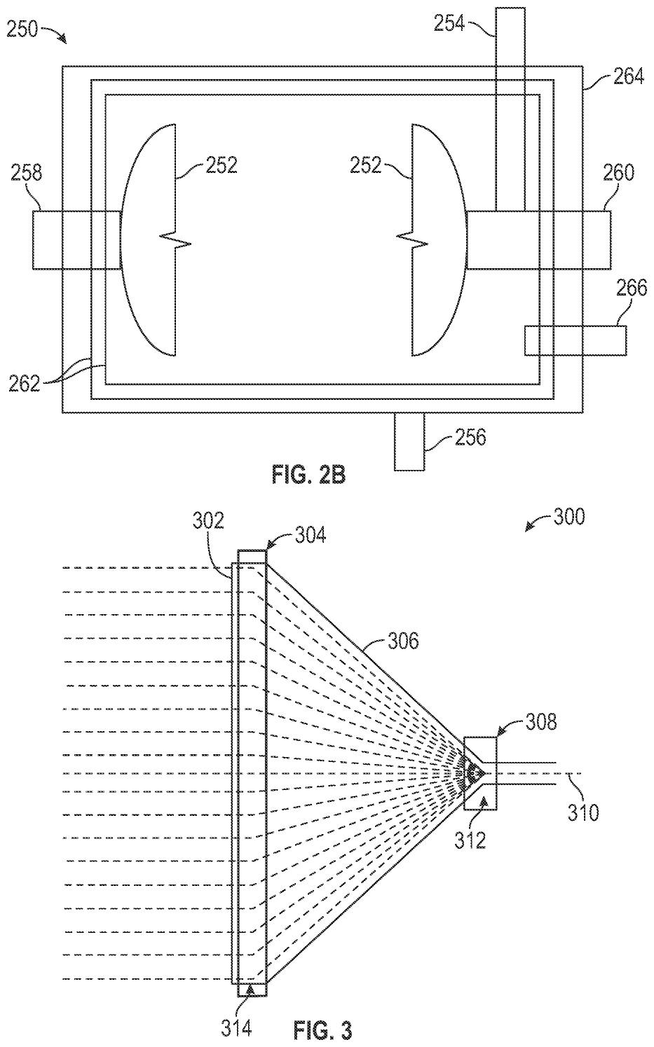

FIG. 2A illustrates a perspective cut-away view of an exemplary 8.5 cell elliptical superconducting RF structure 220 that can also form elements of an electron accelerator adapted for use in accordance with the disclosed embodiments. Note that varying embodiments can employ alternative cavity geometries and/or cell numbers. FIG. 2A generally indicates the operating principles of an elliptical RF cavity. Advancements in SRF technology can enable even more compact and efficient accelerators for associated applications.

The RF structure 220 of FIG. 2A demonstrates the principle of operation in which alternating RF electric fields can be configured to accelerate groups of electrons timed to arrive in each cavity when the electric field in that cavity causes the electrons to gain additional energy. In the particular embodiment shown in FIG. 2A, a voltage generator can induce an electric field within the RF cavity. Its voltage can oscillate, for example, with a radio frequency of 1.3 Gigahertz or 1.3 billion times per second. An electron source 224 can inject particles into the cavity in phase with the variable voltage provided by the voltage generator of the RF structure 220. Arrow(s) 226 shown in FIG. 2A indicate that the electron injection and cavity RF phase is adjusted such that electrons experience or “feel” an average force that accelerates them in the forward direction, while arrow(s) 228 indicate that electrons are not present in a cavity cell when the force is in the backwards direction. The structure 220 can be cooled with a conduction cooling system 222.

It can be appreciated that the example RF structures 100 and 220, respectively shown in FIGS. 1 and 2A, represent examples only and that electron accelerators of other types and configurations/structures/frequencies may be implemented in accordance with alternative embodiments. That is, the disclosed embodiments are not limited structurally to the example electron accelerator structures 100, 220, respectively shown in FIGS. 1 and 2, but represent one possible type of structure that may be employed with particular embodiments. Alternative embodiments may vary in structure, arrangement, frequency, and type of utilized accelerators, RF structures, and so forth.

In certain embodiments, a coupler feeds RF power into the cavity. A vacuum system can be used to evacuate the cavity. In certain embodiments, a cryogenic system, or cryostat, can be used to keep the cavity at very low temperatures. In other embodiments a conducting system can be used to be a cooling system to remove heat generated by the oscillating electric and magnetic fields.

Aspects of such systems are illustrated in FIG. 2B. Specifically, in an embodiment, an accelerator system 250 can include an accelerating cavity 252 with a radio frequency (RF) input 254, and vacuum connection 256. The beam source 258 and beam exit 260 associated with the accelerator cavity (or cavities) 252 provide a respective beam entrance and beam exit through magnetic and thermal shielding layers 262 and a cryostat 264. The cryostat 264 is configured to maintain the temperature of the accelerator system 250 and can be cooled with a cryogenic connection 266.

In order to use X-rays for industrial sterilization and other irradiation processes, an electronic beam from an accelerator as illustrated in FIG. 1, FIG. 2A or 2B, can be used to produce Bremsstrahlung X-rays by directing the electron beam onto the target. U.S. Pat. No. 10,880,984 titled “Permanent Magnet E-Beam/X-Ray Horn” describes such a system. U.S. Pat. No. 10,880,984 is herein incorporated by reference in its entirety. Other such embodiments are detailed in U.S. application Ser. No. 17/116,880 titled ““Permanent Magnet E-Beam/X-Ray Horn”. U.S. application Ser. No. 17/116,880 is herein incorporated by reference in its entirety.

FIG. 3 illustrates a schematic diagram of a magnetic apparatus 300, in accordance with an embodiment. The magnetic apparatus 300 can be used to produce electron beams or X-rays for irradiation processes including, but not limited to industrial sterilization and other irradiation purposes. The magnetic apparatus 300 can include a scanning electromagnet 308 and a vacuum chamber 306. The vacuum chamber 306 can include a first section 312 and a second section 314. The second section 314 can be wider than the first section 312. Note that in some example embodiments, the vacuum chamber 306 may be a cone-shaped vacuum chamber or a horn-shaped vacuum chamber referred to as a scanning horn vacuum chamber. It should be appreciated, however, that the vacuum chamber 306, although shown as horn-shaped, is not limited to such a shape. Other configurations and shapes are possible. For example, the vacuum chamber 306 can be a rectangular or box-shaped vacuum chamber including a scan horn protection assembly as further detailed herein.

The scanning electromagnet 308 can be utilized to redirect a beam of charged particles. Note that from a physics perspective, there is no physical interaction between the scanning electromagnet 308 and the vacuum chamber 306. The “interaction” is actually between the magnetic field and the charged particles. The vacuum chamber 306 keeps the atmosphere from interfering with the charged particles. The vacuum chamber 306 can be configured from materials that are “transparent” to the magnetic field of the magnets that are external the vacuum chamber 306.

Additionally, it can be appreciated that the disclosed embodiments can be implemented for all charged particles. Electrons, however, are approximately 2000 times lighter than the next lightest particle (protons) so an implementation may be presently only practical for electrons.

A beam line 310 is also depicted in FIG. 3 with respect to the scanning electromagnet 308. A parallelizing permanent magnet array 304 is shown in FIG. 3 with respect to the vacuum chamber 306 at a second section 314 of the vacuum chamber 306, and proximate to a target 302, which may be a Bremsstrahlung target or an object that is being irradiated. Note that in some embodiments, the target 302 can be located in a vacuum window if operating in an electron beam mode. It should be understood that some systems may use more than one vacuum window.

The target 302 can also serve in some example embodiments, as both a vacuum window and a Bremsstrahlung target if operating in an X-ray mode. In still other example embodiments, the vacuum window and Bremsstrahlung target can be separate components. If separate, this allows switching between electron beam and X-ray mode by moving the Bremsstrahlung target out of the way. Note that the parallelizing permanent magnet array 304 can be located within or outside the vacuum chamber 306. In certain embodiments, the Bremsstrahlung target 302 can further include cooling elements, which can be air blowers, water channels, or the like, used to manage the heat at the Bremsstrahlung target 302.

It should be appreciated that the disclosed embodiments are not limited to only an X-ray mode. That is, irradiation can use either the electron beam itself or, by way of a Bremsstrahlung converter, X-rays. Thus, to be clear, the disclosed embodiments are not limited to X-rays. A Bremsstrahlung converter can be located after the permanent magnet if used in X-ray mode.

The parallelizing permanent magnet array 304 can be configured from an array of permanent magnets. Note that the strength of a scanning magnet (in this case the electromagnet 308) should be variable in order to produce all the angles necessary to sweep the beam across the target. Thus, an electromagnet may be used as a scanning magnet, which is the case with the scanning electromagnet 308. The required strength of a parallelizing magnet, however, may be proportional to the position of the electron beam from the beam line 310. For this reason, the parallelizing magnet can be configured from permanent magnet materials that do not require an electric current in the context of the parallelizing permanent magnet array 304. The strength of this permanent magnet material is arranged to provide a magnetic field that increases with distance away from the centerline. This configuration can reduce the operating costs of the magnetic apparatus 300 while facilitating the elimination of failure modes in an irradiation facility.

The magnetic apparatus 300 can produce a spatially varying magnetic field so that the electrons are redirected from a diverging pattern to a parallel pattern. That is, the beam can be redirected by the parallelizing permanent magnet array 304 from a diverging pattern output from the scanning electromagnet 308 to a parallel pattern after being subjected to the parallelizing permanent magnet array 304. In some embodiments, the parallelizing permanent magnet array 304 can be configured as an array of permanent magnets. Note that X-rays are not affected by magnetic fields. They must be generated after the electron beam has been parallelized. In other embodiments, the electrons need not be re-parallelized. That is to say, the systems and method disclosed herein can work without re-parallelizing the electrons before they are converted to X-rays, for example.

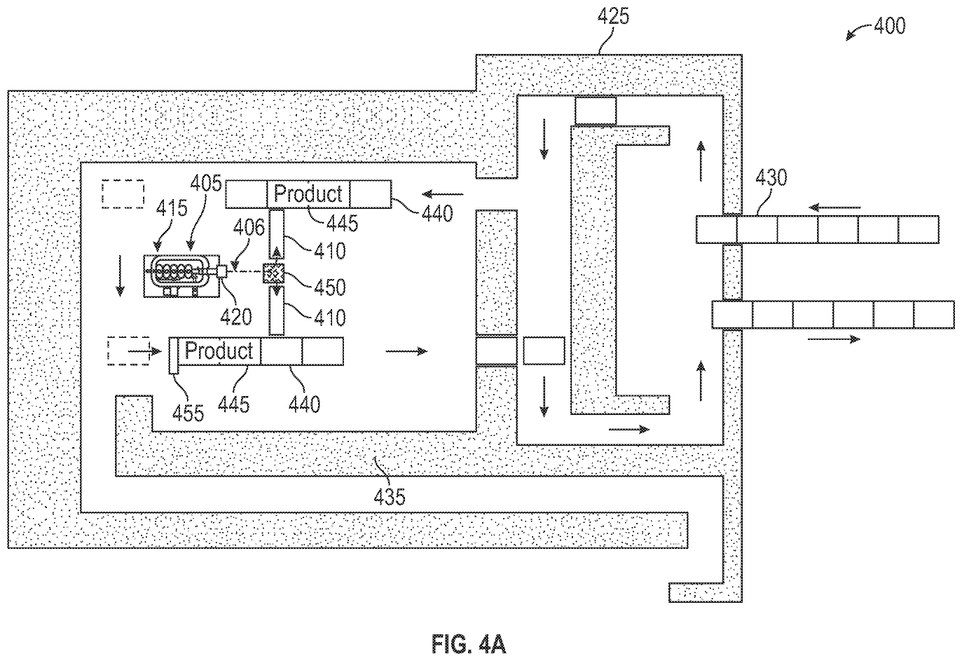

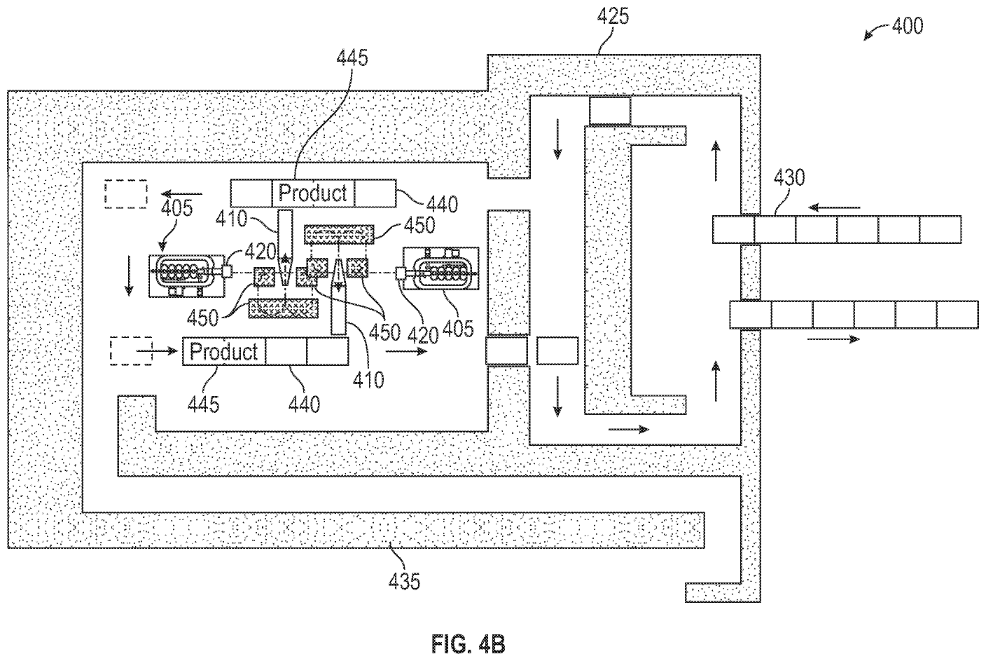

FIG. 4A illustrates an embodiment of a sterilization system 400 using a single accelerator 405 and magnetic apparatus (scan horn), such as magnetic apparatus 300. A treatment facility 425 can comprise a standard medical device sterilization facility. In some cases, this could be a room or a portion of a room at a medical device manufacturing plant, or other such environment. In other embodiments, the treatment facility can comprise a facility for another field, including but not limited to an industrial manufacturing facility, commercial facility, or other such facility where some form of treatment is required.

In some such facilities, a conveyor assembly 430, can be used to convey products 445 (such as medical equipment) along a path so that the equipment can be sterilized. The conveyor assembly 430 moves the material to be sterilized through a radiation field in a precise manner so that the prescribed dosage is delivered to the material. Sterilization of some equipment is accomplished with irradiation. In certain embodiments, the conveyor assembly 430 can include a mechanism to halt the progress of a conveyor assembly, and rotate a product, or pallet of products, at a specific location. This allows the product, or pallet of products, to be irradiated from multiple angles.

The system 400 includes an electron accelerator 405 used to provide an electron beam 406 to two opposed scanning horns 410 (for example, as illustrated in FIG. 3) to provide two-sided e-beam or x-ray irradiation. The arrangement of the accelerator 405 and scanning horns 410 is such that it can fit into an existing enclosure 415 that was previously used, for example, for gamma irradiation. In such embodiments, minimal modifications to the enclosure 415 and conveyor assembly 430 are required.

In certain embodiments, the movement of the material 445 by the conveyor assembly 430 can be monitored, along with the power output of the accelerator system. This can be used to monitor the dose delivered to the material 445 to ensure that the proper dose has been delivered. This can be verified by placing sensors 455 on the material 445 to measure the dose. These sensors 455 readings can be taken after treatment to verify that the proper dose has been delivered. In other embodiments a parametric release is possible, whereby the proper dose is given by virtue of having monitored the proper output of the accelerator 405 and the proper movement by the conveyor system 430. In other embodiments, sensors 455 can comprise stationary sensors that can be placed on the other side of the product 445 to be irradiated, opposite the scan horns 410 to measure the radiation that passes through the product 445 and thereby measure the radiation absorbed by the product 445.

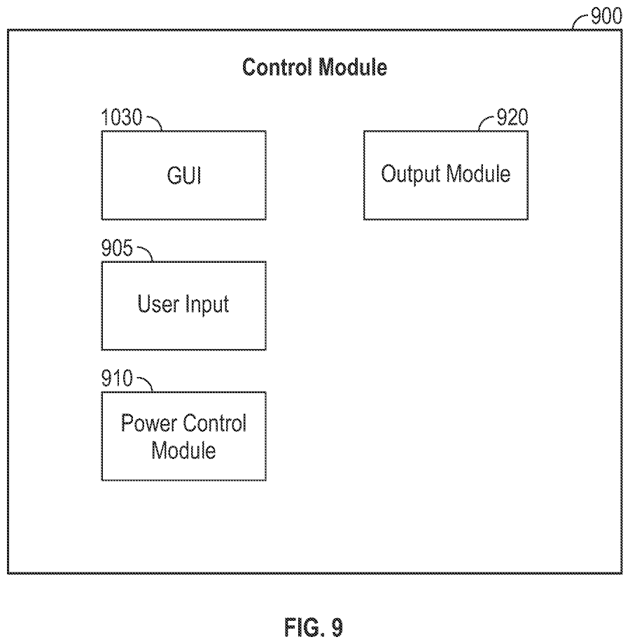

Controlling the dosage is an aspect of the disclosed embodiments. Control of the systems disclosed herein can be accomplished with a control module 900 as illustrated in FIG. 9. By nature, the sterilization process requires product to move along a conveyor assembly, such as conveyor assembly 430. As such, the disclosed embodiments, include a control module 900 to verify the proper dose is applied to the product at the proper time and to ensure the desired sterilization level is reached. The control module 900 can be embodied as software associated with a computer system 1000, and can be configured onboard the disclosed systems or can operate to control such systems via wired or wireless communication.

The control module 900 can include an input module 905 for receiving sterilization or dosing parameters associated with a given product. The control module 900 can provide a user interface 1030 that allows the user to define dosing parameters for the desired application/product. For example, the input module 905 can receive travel path parameters (e.g., a conveyor path) for a product. This can include the physical location of the travel path, along with the speed of product along the travel path. The user can select a desired dosage for the product and input that dosage using the user interface 1030. In certain embodiments, the dosage can be suggested by the control module 900 according to the parameters associated with the product.

The control module 900 can further include a power control module 910. The power control module 910 can use the parameters provided to the control module 900, either as user input 905 entered via GUI 1030, and/or as programmed presets as a part of the power control module 910, to control power of the accelerator 405.

Once the path of the product and power of the beam is determined, an output module 920 associated with the control module 900 can provide instructions to the accelerator assembly 405 to manage the power of the beam to ensure the product is properly dosed. The output module 920 can provide wired or wireless output to the accelerator assembly 405.

Because the system 400 includes the use of irradiation, the walls 435 and/or shielding of the conveyor assembly (which can include a conveyor belt, driving motor, and the like) can be configured of a sufficient amount of shielding material to ensure the radiation is contained as prescribed by applicable regulations, and that treatment is targeted to the desired treatment areas 440. The shielding can further prevent unintended irradiation of external environments.

As illustrated, in FIG. 4A, a deflector 420 and bend magnet(s) 450 can be used to deflect electron beam 406 from accelerator 405 to two scan horns 410. The scan horns 410 are used to, optionally, convert the electron beam 406 into an X-Ray beam, and to scan a product path at the treatment areas 440 in a manner necessary to treat the product 445.

FIG. 4B illustrates another embodiment of a system 400; this arrangement making use of multiple accelerators 405. In such an embodiment, a series of beam diverters embodied as bend magnets 450 can be used to direct beam 406 to multiple scan horns 410. The use of multiple accelerators 405 and scan horns 410 increases the throughput of the associated system.

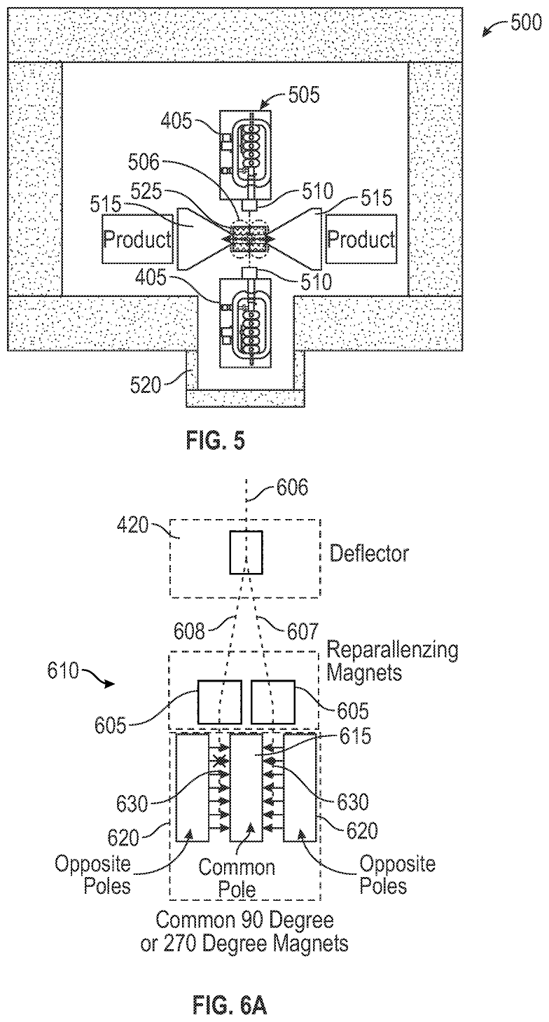

FIG. 5 illustrates another embodiment of a treatment system 500, with one or more accelerators 505 arranged to be vertically orientated. In the embodiment, an accelerator 505 can be housed in a facility housing 520. In certain embodiments, the facility housing 520 can comprise a former Cobalt storage pool, or other such retrofit location. Deflectors 510 and bend magnets 525 associated with the accelerators 505 can direct beam 506 to scan horns 515. The scan horns 515 can be used to divert irradiation onto product for sterilization, as in other embodiments.

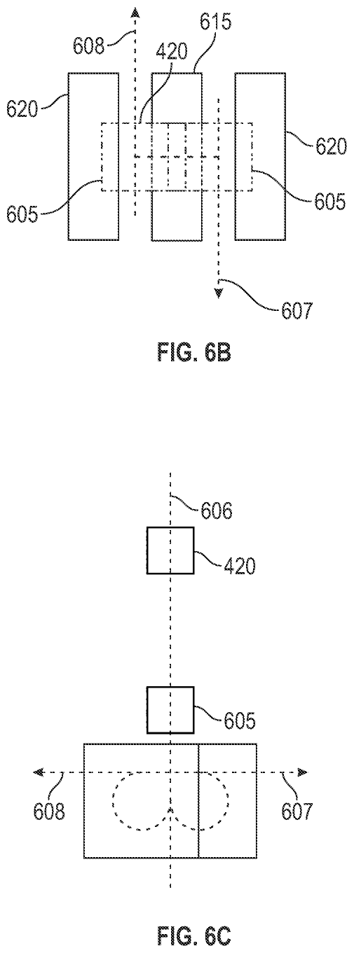

FIGS. 6A-6C illustrate aspects of the assembly, including aspects of the deflector 420 in FIG. 6A. The deflector can split a beam such as electron beam 606 into two beams, electron beam 607 and electron beam 608 respectively. The system can alternate between the two beam paths. Re-parallelizing magnets 605 can be used to alter the beam path of each respective beam, so that it can enter a beam bending assembly 610.

The beam bending assembly 610 is used to alternatively bend or redirect a beam either 90 degrees or 270 degrees respectively. The beam bending assembly 610 includes a common pole magnet 615 in between two opposite pole magnets 620. The field 630 between the common pole magnet 615 and opposite pole magnets 620 can be used to redirect the beam 90 degrees from incidence on one side and 270 degrees from incidence on the other.

Using the beam bending assembly 610 a single accelerator, such as accelerator 405, can be used to send beam 606 in two opposite directions for treatment, in accordance with, for example, the single accelerator embodiment, illustrated in FIG. 4A, or for multiple accelerator embodiments, as illustrated in FIG. 4B.

This arrangement is further illustrated in elevation in FIG. 6B. As illustrated the incident beam is split into electron beam 606 and electron beam 607, which propagate in opposite directions.

FIG. 6C. illustrates a plan view of the arrangement, showing that the incident beam 606 is split into beam 607 directed in one direction and beam 608 directed in the opposite direction.

It should be appreciated that the angles provided herein are exemplary. In other embodiments, any angular value can be used as necessary to direct the beam from the accelerator to the scan horn. For embodiments that use the common pole design as shown then the angles need to be the same. In application where the angles need to be different, the bend magnets 450 need to be separated, each with their own North and South poles. 270 degrees offers some advantages, but is not required. It should further be appreciated that the drawings do not include other focusing elements such as quadrupoles and solenoids that may be used to provide transport from the accelerator to the scan horn.

The X-ray horn as illustrated in FIG. 3, requires vacuum to operate optimally. Although rare, failure of the window between the vacuum chamber and the ambient environment is catastrophic to the accelerator. Specifically, the release of the vacuum in the event of a breach would introduce debris and extreme pressure, which could be highly damaging to the accelerator. Automatic valves are generally not fast enough to close off the accelerator from the scan horn in the event of a large vacuum failure.

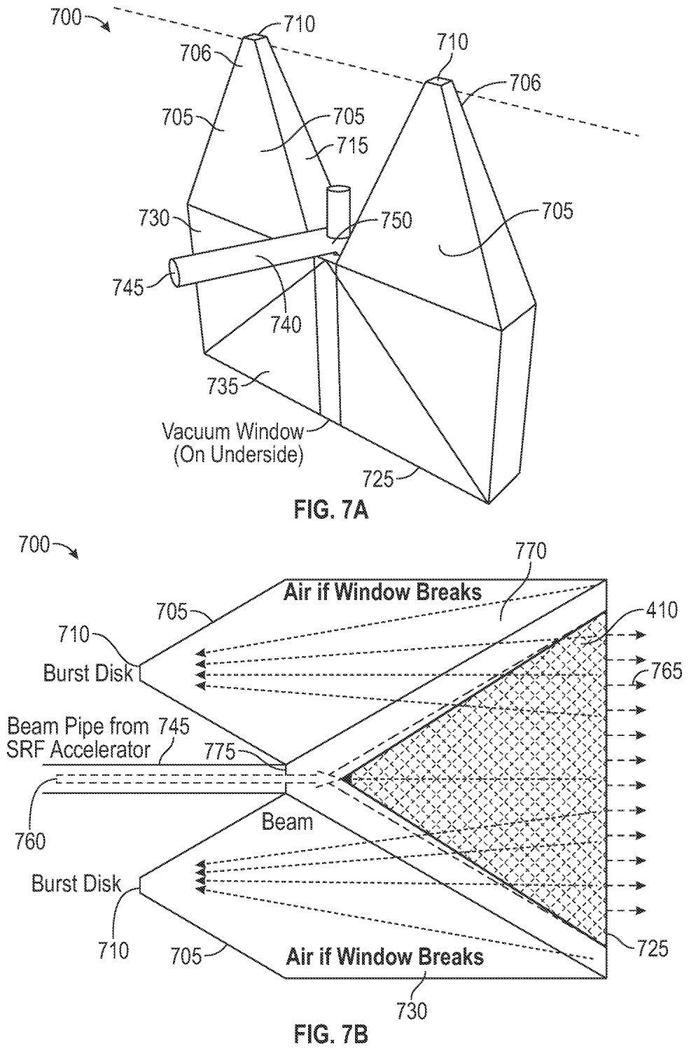

Thus, in certain embodiments, a scan horn protector assembly 700 as illustrated in FIG. 7A and FIG. 7B, can be used to minimize the impact on the system in case of such a failure. In the event of a vacuum window 720 failure, a shock wave of atmospheric pressure will rush from the point of the rupture to fill the vacuum chamber 730.

Aspects of the scan horn protector assembly 700 include the vacuum chamber housing 735, creating a vacuum chamber 730. The assembly 700 has a proportionally small aperture of the beam port 740 relative to the area of the shock wave. The amount of atmosphere and debris entering the beam port 740 is reduced by this ratio.

Aspects further include shock wave concentrator cones 705, configured as a part of the vacuum chamber housing 735, with sacrificial burst discs 710 at the end 706 of the concentrator cones 705. The shape of the shock wave concentrator cones 705 are configured to direct shock waves away from the fragile aspects of the accelerator assembly. Likewise, the sacrificial burst discs 710 are configured to break in the event of a sudden pressure change, to alleviate the associated pressure reflections that would otherwise be transferred to the fragile aspects of the accelerator assembly.

In addition, the beam pipe 745 can include an angle 750 forming a heavy debris trap 715 to prevent large debris from entering the beam pipe 745. The beam in the beam pipe 745 can be configured to bend with a beam bending magnet. The beam bending magnet can be selected to ensure the beam travels in the beam pipe 745. The beam pipe angle 750 can be selected to be 90 degrees in certain embodiments, although other angles will also suffice for trapping certain debris.

FIG. 7B illustrates the dynamics of the scan horn protector assembly 700 in accordance with the disclosed embodiments. As illustrated a scan horn 410 can be disposed inside the housing 735. During normal operation, the beam 760 travels through the beam pipe 745 to the scan horn 410 which can the beam through the vacuum 730 where it is parallelized as illustrated by beams 765 exiting the window 725.

Because the chamber is under vacuum, if the window 725 breaks, airflow 770 will rush into the volume creating a shockwave. The proportionally small aperture 775 of the beam pipe relative to the area of the shock wave will reduce the impact of the shockwave on the accelerator. Likewise, the sacrificial burst discs 710 at the end of the concentrating cones 705, will break and release pressure, further protecting the accelerator assembly from damage.

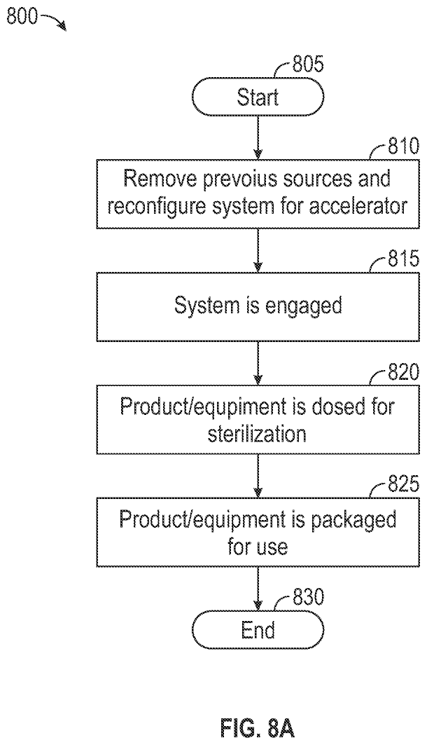

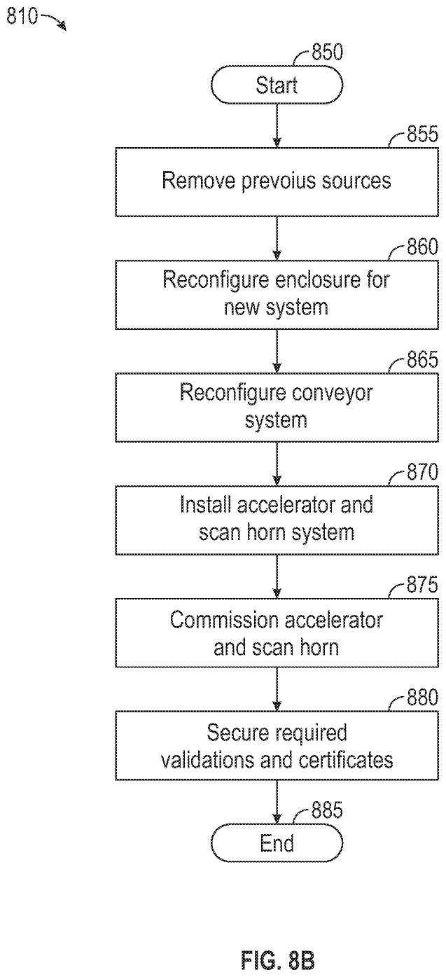

FIG. 8A illustrates a method 800 for retrofitting a facility with sterilization equipment, and thereafter sterilizing equipment in accordance with the embodiments provided herein. It should be appreciated that in an exemplary embodiment, the systems disclosed herein can be used for sterilizing medical equipment in accordance with this method. However, in other embodiments, the system can be used for sterilization of other devices or equipment. The method begins at step 805.

At step 810, previous irradiation sources, including but not limited to cobalt sources, can be removed from a facility. The cobalt enclosure can be reconfigured to support a new system, and one or more accelerator cavities and associated scan horns can be installed in the facility as illustrated. The accelerator assembly can be positioned with scan horns configured to direct beam onto a product treatment area. Aspects of step 810 are illustrated in FIG. 8B.

Next at step 815, the conveyor assembly can be engaged, with product being transported into product treatment areas, for example with a conveyor belt or other such transport assembly. The product (e.g., medical equipment) can be dosed with a desired amount of irradiation in order to sterilize the equipment at step 820. The system can then transport the product to a facility exit where it can optionally be checked to verify it has been sufficiently dosed, and then packaged for use at step 825. The method then ends at 830.

FIG. 8B illustrates aspects of the reconfiguration of the facility for the new system, at step 810. The reconfiguration step 810 begins at 850. At step 855 previous radiation sources can be removed from the sterilization assembly. Proper safety protocols should be taken as these sources are removed. Next at 860 the enclosure for the source (e.g., a cobalt source) can be reconfigured to support new systems. Likewise at step 865 the conveyor system can be reconfigured or replaced as necessary.

With the facility prepared, the accelerator and scan horn system can be installed at step 870. The accelerator and scan horn systems can be commissioned at step 875. Next at step 880, the required facility validations and certifications can be performed. At this point the system is ready for engagement as shown at step 815 and the reconfiguration step 810 ends at step 885.

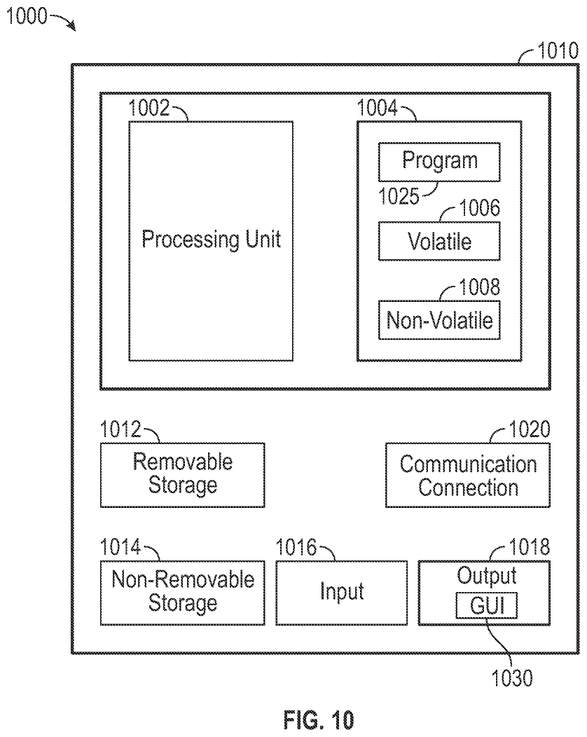

FIGS. 10-12 are provided as exemplary diagrams of data-processing environments in which embodiments of the present invention may be implemented. It should be appreciated that FIGS. 10-12 are only exemplary and are not intended to assert or imply any limitation with regard to the environments in which aspects or embodiments of the disclosed embodiments may be implemented. Many modifications to the depicted environments may be made without departing from the spirit and scope of the disclosed embodiments.

A block diagram of a computer system 1000 that executes programming for implementing parts of the methods and systems disclosed herein is shown in FIG. 10. A computing device in the form of a computer 1010 configured to interface with sensors, peripheral devices, and other elements disclosed herein may include one or more processing units 1002, memory 1004, removable storage 1012, and non-removable storage 1014. Memory 1004 may include volatile memory 1006 and non-volatile memory 1008. Computer 1010 may include or have access to a computing environment that includes a variety of transitory and non-transitory computer-readable media such as volatile memory 1006 and non-volatile memory 1008, removable storage 1012 and non-removable storage 1014. Computer storage includes, for example, random access memory (RAM), read only memory (ROM), erasable programmable read-only memory (EPROM) and electrically erasable programmable read-only memory (EEPROM), flash memory or other memory technologies, compact disc read-only memory (CD ROM), Digital Versatile Disks (DVD) or other optical disk storage, magnetic cassettes, magnetic tape, magnetic disk storage, or other magnetic storage devices, or any other medium capable of storing computer-readable instructions as well as data including image data.

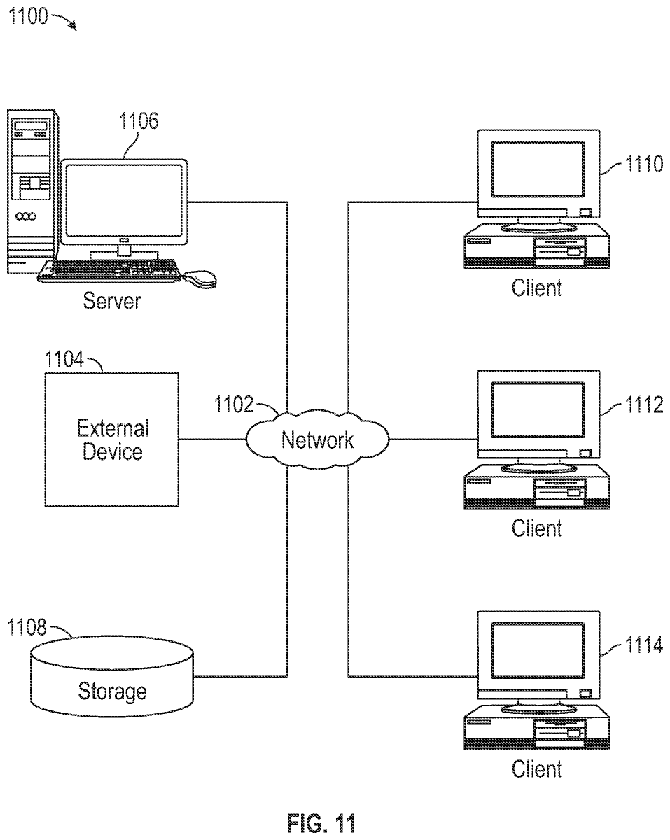

Computer 1010 may include or have access to a computing environment that includes input 1016, output 1018, and a communication connection 1020. The computer may operate in a networked environment using a communication connection 1020 to connect to one or more remote computers, remote sensors, detection devices, hand-held devices, multi-function devices (MFDs), mobile devices, tablet devices, mobile phones, Smartphones, or other such devices. The remote computer may also include a personal computer (PC), server, router, network PC, RFID enabled device, a peer device or other common network node, or the like. The communication connection may include a Local Area Network (LAN), a Wide Area Network (WAN), Bluetooth connection, or other networks. This functionality is described more fully in the description associated with FIG. 11 below.

Output 1018 is most commonly provided as a computer monitor, but may include any output device. Output 1018 and/or input 1016 may include a data collection apparatus associated with computer system 1000. In addition, input 1016, which commonly includes a computer keyboard and/or pointing device such as a computer mouse, computer track pad, or the like, allows a user to select and instruct computer system 1000. A user interface can be provided using output 1018 and input 1016. Output 1018 may function as a display for displaying data and information for a user, and for interactively displaying a graphical user interface (GUI) 1030.

Note that the term “GUI” generally refers to a type of environment that represents programs, files, options, and so forth by means of graphically displayed icons, menus, and dialog boxes on a computer monitor screen. A user can interact with the GUI to select and activate such options by directly touching the screen and/or pointing and clicking with a user input device 1016 such as, for example, a pointing device such as a mouse and/or with a keyboard. A particular item can function in the same manner to the user in all applications because the GUI provides standard software routines (e.g., module 1025) to handle these elements and report the user's actions. The GUI can further be used to display the electronic service image frames as discussed below.

Computer-readable instructions, for example, program module or node 1025, which can be representative of other modules or nodes described herein, are stored on a computer-readable medium and are executable by the processing unit 1002 of computer 1010. Program module or node 1025 may include a computer application. A hard drive, CD-ROM, RAM, Flash Memory, and a USB drive are just some examples of articles including a computer-readable medium.

FIG. 11 depicts a graphical representation of a network of data-processing systems 1100 in which aspects of the present invention may be implemented. Network data-processing system 1100 is a network of computers or other such devices such as mobile phones, smartphones, sensors, detection devices, controllers, and the like in which embodiments of the present invention may be implemented. Note that the system 1100 can be implemented in the context of a software module such as program module 1025. The system 1100 includes a network 1102 in communication with one or more clients 1110, 1112, and 1114. Network 1102 may also be in communication with one or more device 1104, servers 1106, and storage 1108. Network 1102 is a medium that can be used to provide communications links between various devices and computers connected together within a networked data processing system such as computer system 1000. Network 1102 may include connections such as wired communication links, wireless communication links of various types, fiber optic cables, quantum, or quantum encryption, or quantum teleportation networks, etc. Network 1102 can communicate with one or more servers 1106, one or more external devices such as a controller, actuator, sensor, or other such device 1104, and a memory storage unit such as, for example, memory or database 1108. It should be understood that device 1104 may be embodied as a detector device, microcontroller, controller, receiver, transceiver, or other such device.

In the depicted example, device 1104, server 1106, and clients 1110, 1112, and 1114 connect to network 1102 along with storage unit 1108. Clients 1110, 1112, and 1114 may be, for example, personal computers or network computers, handheld devices, mobile devices, tablet devices, smartphones, personal digital assistants, microcontrollers, recording devices, MFDs, etc. Computer system 1000 depicted in FIG. 10 can be, for example, a client such as client 1110 and/or 1112.

Computer system 1000 can also be implemented as a server such as server 1106, depending upon design considerations. In the depicted example, server 1106 provides data such as boot files, operating system images, applications, and application updates to clients 1110, 1112, and/or 1114. Clients 1110, 1112, and 1114 and external device 1104 are clients to server 1106 in this example. Network data-processing system 1100 may include additional servers, clients, and other devices not shown. Specifically, clients may connect to any member of a network of servers, which provide equivalent content.

In the depicted example, network data-processing system 1100 is the Internet with network 1102 representing a worldwide collection of networks and gateways that use the Transmission Control Protocol/Internet Protocol (TCP/IP) suite of protocols to communicate with one another. At the heart of the Internet is a backbone of high-speed data communication lines between major nodes or host computers consisting of thousands of commercial, government, educational, and other computer systems that route data and messages. Of course, network data-processing system 1100 may also be implemented as a number of different types of networks such as, for example, an intranet, a local area network (LAN), or a wide area network (WAN). FIGS. 10 and 11 are intended as examples and not as architectural limitations for different embodiments of the present invention.

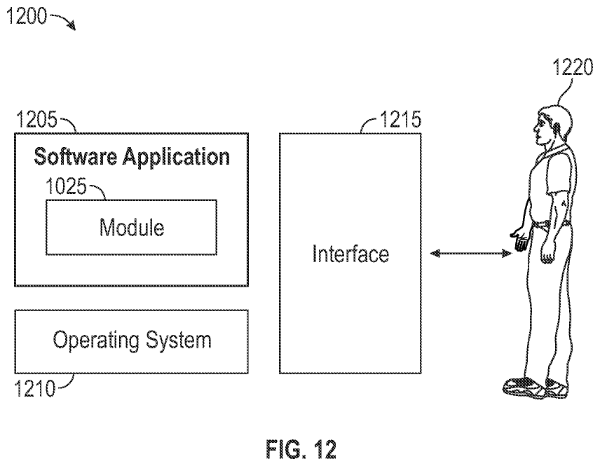

FIG. 12 illustrates a software system 1200, which may be employed for directing the operation of the data-processing systems such as computer system 1000 depicted in FIG. 10. Software application 1205, may be stored in memory 1004, on removable storage 1012, or on non-removable storage 1014 shown in FIG. 10, and generally includes and/or is associated with a kernel or operating system 1210 and a shell or interface 1215. One or more application programs, such as module(s) or node(s) 1025, may be “loaded” (i.e., transferred from removable storage 1014 into the memory 1004) for execution by the data-processing system 1000. The data-processing system 1000 can receive user commands and data through user interface 1215, which can include input 1016 and output 1018, accessible by a user 1220. These inputs may then be acted upon by the computer system 1000 in accordance with instructions from operating system 1210 and/or software application 1205 and any software module(s) 1025 thereof.

Generally, program modules (e.g., module 1025) can include, but are not limited to, routines, subroutines, software applications, programs, objects, components, data structures, etc., that perform particular tasks or implement particular abstract data types and instructions. Moreover, those skilled in the art will appreciate that elements of the disclosed methods and systems may be practiced with other computer system configurations such as, for example, hand-held devices, mobile phones, smart phones, tablet devices, multi-processor systems, printers, copiers, fax machines, multi-function devices, data networks, microprocessor-based or programmable consumer electronics, networked personal computers, minicomputers, mainframe computers, servers, medical equipment, medical devices, and the like.

Note that the term module or node as utilized herein may refer to a collection of routines and data structures that perform a particular task or implements a particular abstract data type. Modules may be composed of two parts: an interface, which lists the constants, data types, variables, and routines that can be accessed by other modules or routines; and an implementation, which is typically private (accessible only to that module), and which includes source code that actually implements the routines in the module. The term module may also simply refer to an application such as a computer program designed to assist in the performance of a specific task such as word processing, accounting, inventory management, etc., or a hardware component designed to equivalently assist in the performance of a task.

The interface 1215 (e.g., a graphical user interface 1030) can serve to display results, whereupon a user 1220 may supply additional inputs or terminate a particular session. In some embodiments, operating system 1210 and GUI 1030 can be implemented in the context of a “windows” system. It can be appreciated, of course, that other types of systems are possible. For example, rather than a traditional “windows” system, other operation systems such as, for example, a real time operating system (RTOS) more commonly employed in wireless systems may also be employed with respect to operating system 1210 and interface 1215. The software application 1205 can include, for example, module(s) 1025, which can include instructions for carrying out steps or logical operations such as those shown and described herein.

The following description is presented with respect to embodiments of the present invention, which can be embodied in the context of, or require the use of a data-processing system such as computer system 1000, in conjunction with program module 1025, and data-processing system 1100 and network 1102 depicted in FIGS. 10-12. The present invention, however, is not limited to any particular application or any particular environment. Instead, those skilled in the art will find that the systems and methods of the present invention may be advantageously applied to a variety of system and application software including database management systems, word processors, and the like. Moreover, the present invention may be embodied on a variety of different platforms including Windows, Macintosh, UNIX, LINUX, Android, Arduino, and the like. Therefore, the descriptions of the exemplary embodiments, which follow, are for purposes of illustration and not considered a limitation.

Based on the foregoing, it can be appreciated that a number of example embodiments, preferred and alternative, are disclosed herein. In an embodiment, a treatment system, comprises a particle accelerator configured to produce a beam of charged particles, a deflector configured to redirect the beam of charged particles, a vacuum chamber that prevents the atmosphere from interfering with the charged particles, and a magnet array for directing the beam of charged particles onto a product, wherein the beam of charged particles is used to sterilize the product.

In an embodiment, the beam of charged particles comprises an electron beam. In an embodiment, the beam of charged particles comprises an X-ray beam. In an embodiment, the treatment system further comprises a target material, wherein the beam of charged particles from the particle accelerator is directed onto the target material to produce Bremsstrahlung X-rays.

In an embodiment, the magnet array comprises a parallelizing magnet array for parallelizing the beam of charged particles.

In an embodiment, the treatment system further comprises a scan horn protector assembly. The scan horn protector assembly comprises a housing, a concentrator cone, and a sacrificial burst disc at the end of the concentrator cone.

In an embodiment of the treatment system, the charged particles are directed onto at least one treatment area.

In an embodiment, a system comprises a particle accelerator configured to produce a beam of electrons, a deflector configured to split the electron beam into a first beam and a second beam, a first re-parallelizing magnet configured to redirect the first beam into a beam bending assembly, wherein the beam bending assembly directs the first beam in a first direction, and a second re-parallelizing magnet configured to redirect the second beam into the beam bending assembly wherein the beam bending assembly directs the second beam in a second direction.

In an embodiment, the beam bending assembly comprises a common pole magnet in spaced relation between two opposite pole magnets. In an embodiment, a magnetic field exists between the common pole magnet and opposite pole magnets. In an embodiment, the first direction of the first beam is different from the second direction of the second beam.

In an embodiment, the first direction of the first beam is directed to the path of travel of a product on a conveyor assembly and the second direction of the second beam is directed to a different path of travel of a product on the conveyor assembly. In an embodiment, the product comprises a medical product.

In an embodiment the system further comprises a target material, wherein the beam of charged particles from the particle accelerator is directed onto the target material to produce Bremsstrahlung X-rays.

In another embodiment, a treatment method comprises producing a particle beam with a particle accelerator, splitting the particle beam into a first particle beam and a second particle beam with a deflector, redirecting the first beam into a beam bending assembly, with a first re-parallelizing magnet, redirecting the second beam into a beam bending assembly with a second re-parallelizing magnet, directing the first particle beam onto a treatment area with a beam bending assembly, and directing the second particle beam onto a different treatment area with the beam bending assembly.

In an embodiment, the particle beam comprises an electron beam. In an embodiment, the treatment method comprises producing an x-ray beam by directing the electron beam onto a target.

In an embodiment, the treatment method further comprises conveying a product through the treatment area wherein the particle beam is incident on the product. In an embodiment of the method the product comprises a medical product.

It will be appreciated that variations of the above-disclosed and other features and functions, or alternatives thereof, may be desirably combined into many other different systems or applications. It will also be appreciated that various presently unforeseen or unanticipated alternatives, modifications, variations, or improvements therein may be subsequently made by those skilled in the art which are also intended to be encompassed by the following claims.

What is claimed is:

1. A treatment system, comprising:
a particle accelerator configured to produce a beam of charged particles;
a deflector configured to redirect the beam of charged particles;
a vacuum chamber that prevents atmosphere from interfering with the beam of charged particles; and
a magnet array for directing the beam of charged particles onto a product, wherein the beam of charged particles is used to sterilize the product.

2. The treatment system of claim 1 wherein the beam of charged particles comprises an electron beam.

3. The treatment system of claim 1 wherein the beam of charged particles comprises an X-ray beam.

4. The treatment system of claim 1 further comprising:
a target material, wherein the beam of charged particles from the particle accelerator is directed onto the target material to produce Bremsstrahlung X-rays.

5. The treatment system of claim 1 wherein the magnet array comprises:
a parallelizing magnet array for parallelizing the beam of charged particles.

6. The treatment system of claim 1 further comprising:
a scan horn protector assembly.

7. The treatment system of claim 6 wherein the scan horn protector assembly comprises:
a housing;
a concentrator cone; and
a sacrificial burst disc at an end of the concentrator cone.

8. The treatment system of claim 1 wherein the charged particles are directed onto at least one treatment area.

9. A system, comprising:
a particle accelerator configured to produce a beam of electrons;
a deflector configured to split the beam of electrons into a first beam and a second beam;
a first re-parallelizing magnet configured to redirect the first beam into a beam bending assembly, wherein the beam bending assembly directs the first beam in a first direction; and
a second re-parallelizing magnet configured to redirect the second beam into the beam bending assembly wherein the beam bending assembly directs the second beam in a second direction.

10. The system of claim 9 wherein the beam bending assembly comprises:
a common pole magnet in spaced relation between two opposite pole magnets.

11. The system of claim 10 wherein a magnetic field exists between the common pole magnet and opposite pole magnets.

12. The system of claim 9 further comprising:
a target material, wherein the beam of electrons from the particle accelerator is directed onto the target material to produce Bremsstrahlung X-rays.

13. The system of claim 9 wherein the first direction of the first beam is different from the second direction of the second beam.

14. The system of claim 9 wherein the first direction of the first beam is directed to a path of travel of a product on a conveyor assembly; and
Wherein the second direction of the second beam is directed to a different path of travel of a product on the conveyor assembly.

15. The system of claim 14 wherein the product comprises a medical product.

16. A treatment method comprising:
producing a particle beam with a particle accelerator;
splitting the particle beam into a first particle beam and a second particle beam with a deflector;
redirecting the first particle beam into a beam bending assembly, with a first re-parallelizing magnet;
redirecting the second particle
beam into a beam bending assembly with a second re-parallelizing magnet;
directing the first particle beam onto a treatment area with a beam bending assembly; and
directing the second particle beam onto a different treatment area with the beam bending assembly.

17. The treatment method of claim 16 wherein the particle beam comprises an electron beam.

18. The treatment method of claim 17 further comprising:
producing an x-ray beam by directing the electron beam onto a target.

19. The treatment method of claim 16 further comprising:
conveying a product through the treatment area wherein the particle beam is incident on the product.

20. The treatment method of claim 19 wherein the product comprises a medical product.
